(19) United States
(12) Patent Application Publication
(10) Pub. No.: US 2014/0257512 A1
(43) Pub. Date: Sep. 11, 2014

(54) Cementless Hip Resurfacing Prosthesis

(71) Applicant: Lichu Liu, Maumelle, AR (US)

(72) Inventor: Lichu Liu, Maumelle, AR (US)

(21) Appl. No.: 14/198,769

(22) Filed: Mar. 6, 2014

Related U.S. Application Data
(60) Provisional application No. 61/774,064, filed on Mar. 7, 2013.

(51) Int. Cl. A61F 2/36

(52) U.S. Cl. CPC A61F 2/3603
USPC 623/23.14

(57) ABSTRACT

A cementless hip resurfacing prosthesis is provided. The prosthesis comprises a metallic cap that is fitted over the individual's femoral head to provide a new acetabular articulating surface. The metallic cap is affixed to the femur via a locking means extending from the inner surface of the metallic cap, which engages with a complimentary tubular fixation means attached to a plate. The plate is affixed to the lateral surface of the femur via a fixation screw, which extends through the fixation means, through the locking means, and engages with a complimentary threaded portion within the locking means. The plate is further affixed to the femur via a plurality of cortical screws, which also act to distribute the forces from the hip joint to avoid stress shielding and bone resorption effects commonly associated with hip replacement prostheses. Furthermore, the present invention is designed to reduce shear stresses on the femoral neck.

### CROSS REFERENCE TO RELATED APPLICATION

This application claims the benefit of U.S. Provisional Application No. 61/774,064 filed on Mar. 7, 2013 entitled “Cementless Hip Resurfacing Prosthesis.” The above identified patent application is herein incorporated by reference in its entirety to provide continuity of disclosure.

### BACKGROUND OF THE INVENTION

1. Field of the Invention

The present invention relates to hip arthroplasty. More specifically, the present invention relates to a femoral head resurfacing prosthesis.

Resurfacing of the femoral head has emerged in recent years as an alternative to more traditional total hip replacement surgeries. Hip resurfacing is generally suitable for younger patients that do not have an inflammatory joint disease and are not allergic to the metals commonly used in such resurfacing prostheses. Resurfacing of the hip joint utilizes two separate components, a femoral head prosthesis and a corresponding prosthetic acetabular articulating surface. The femoral head prosthesis is affixed over the natural head of the femur, providing a barrier between the acetabular articulating surface and the natural femoral head to reduce wear and tear on the natural femoral head. A corresponding metal surface is also placed within the patient's acetabulum. The two new articulating surfaces in the patient's hip joint protect the bones from further degradation.

Hip resurfacing has several potential advantages over traditional total hip replacement procedures: hip resurfacing requires less bone to be removed than traditional total hip replacement, there is a smaller chance of hip dislocation because hip resurfacing uses the patient's natural femoral head, which is larger than the heads of prostheses traditionally used in total hip replacement surgery, revision surgery is more effective because more bone is left available for the revision surgeon, and surgeon-induced leg length differences are also less common. Total hip replacement involves removing the entire femoral head and neck regions and placing a femoral stem prosthesis within the exposed femoral canal, which articulates against a corresponding cup that is placed within the acetabulum. The main difference between hip resurfacing and total hip replacement procedures is the femoral component, because the acetabular components for each of the procedures are very similar. Although hip resurfacing is not a minimally invasive procedure, it is still less invasive because it preserves much more of the patient's natural bone and is thus an attractive alternative for suitable patients.

Current hip arthroplasty prostheses have a wide range of problems. Total hip replacement takes much more of the patient's bone away, making the procedure and its associated prostheses potentially undesirable compared to the hip resurfacing procedure. Hip resurfacing prostheses come in cemented and cementless variations. Cemented resurfacing prostheses have two main issues. First, cement has a substantial failure rate for long-term hip arthroplasty prostheses. Second, the curing process of the cement is an exothermic reaction and the heat generated therefrom may damage the femoral head, potentially leading to fractures or avascular necrosis of the femoral head.

The present invention provides a new and improved cementless hip resurfacing prosthesis that comprises a metallic cap adapted to be secured to the outer surface of the femoral head, a cannulated fixation means extending from the metallic cap through the femoral head and neck, a screw designed to be placed through the fixation means and lock said metallic cap and fixation means together, and a femoral plate to provide additional stabilization and body load distribution. The metallic cap further has a porous surface, allowing for osseointegration to occur, which assists in securing the device to the anatomy of the femoral head. The present invention provides a hip resurfacing prosthesis that does not use cement and which is securely held in place against the femoral head, preventing further degradation of the articulating surface.

2. Description of the Prior Art

Devices have been disclosed in the prior art that relate to hip arthroplasty. These include devices that have been patented and published in patent application publications. These devices generally relate to hip stem prostheses or hip resurfacing prostheses. The following is a list of devices deemed most relevant to the present disclosure, which are herein described for the purposes of highlighting and differentiating the unique aspects of the present invention, and further highlighting the drawbacks existing in the prior art.

Several types of femoral stem prostheses used in total hip replacement procedures currently exist in the prior, some of which include U.S. Pat. No. 4,532,661 to Halpern, U.S. Pat. No. 4,718,916 to Morscher, U.S. Pat. No. 4,944,761 to Stuhmer, U.S. Pat. No. 5,108,450 to Horber, U.S. Pat. No. 5,171,323 to Willert, U.S. Pat. No. 5,314,494 to Huiskes, and U.S. Pat. No. 7,914,584 to Bigsby. Although total hip replacement surgeries are very effective at improving patients' outcomes and are generally suitable for almost all types of patients, it is nonetheless an incredibly invasive procedure that removes a substantial amount of the patient's femoral bone. Although a hip resurfacing procedure is not a minimally invasive procedure, it is still less invasive than a total hip replacement, since more of the patient's bone is left intact, and therefore may be a better option for qualifying patients. In total hip replacement procedures, the surgeon performs an ostectomy of the femoral head and neck portions, whereas in hip resurfacing procedures a protective cap is placed over the patient's nature femoral head.

Another such device is U.S. Pat. No. 5,755,809 to Cohen, which discloses a femoral head core channel filling prosthesis. Cohen provides a prosthesis that is drilled into an avascular necrotic region of bone and has a porous filling member for providing internal support to the bone and allowing for ingrowth of healthy, vascularized bone tissue. Although the Cohen device does not strictly deal with hip arthroplasty, it can be used to strengthen the femoral head region of the bone and avoid a potentially necessary total hip replacement or hip resurfacing procedure. The present invention also has a hollow channel section that extends through the head and shaft portions of the femur, but the tubular portions of the present invention's fixation means and locking means are provided to allow a fixation screw to pass therethrough, securely holding the metallic cap against the surface of the femoral head, not for osseointegration.

Yet another such device is U.S. Published Patent Application Publication No. 2009/0048681, which discloses a femoral head metallic shell for a hip resurfacing procedure. The femoral head metallic shell is secured to the surface of the patient's femoral head through a preload tension wire that extends from the femoral shell and through the femoral head and neck to where it exits from the greater trochanter and is connected to a nut-washer combination. The wire may be tightened to ensure that the femoral shell is securely held against the femoral head. The inner surface of the metallic shell is porous in order to promote osseointegration with the metallic shell. The present invention also comprises a metallic shell portion held firmly against the femoral head by a means extending through the femoral head and neck, but the lateral end of the fixation means is secured against the cortical bone of the femur with a plate, which provides additional stability.

The present invention is a new and improved hip resurfacing prosthesis that provides a metallic cap having a porous or roughened inner surface that is affixed to the patient's femoral head to protect the femoral head from further degradation and encourage osseointegration therewith. The metallic cap articulates with a corresponding metallic acetabular prosthetic surface. The metallic cap is held firmly against the femoral head by a cannulated fixation means, which extends through the femoral head and neck to where it exits the greater trochanter or just below the greater trochanter. A fixation screw is inserted through the cannulated fixation means and is tightened to secure the fixation means and the metallic cap together, thereby holding the metallic cap firmly in place. A femoral plate is further provided for increased stability and is held against the cortical bone by the fixation screw and a plurality of cortical screws. It substantially diverges in design elements from the prior art and consequently it is clear that there is a need in the art for an improvement to existing resurfacing prostheses devices. In this regard the instant invention substantially fulfills these needs.

### SUMMARY OF THE INVENTION

In view of the foregoing disadvantages inherent in the known types of hip arthroplasty prostheses now present in the prior art, the present invention provides a new hip resurfacing prosthesis wherein the same can be utilized for providing convenience for the user when undergoing corrective hip joint procedures.

It is therefore an object of the present invention to provide a new and improved hip resurfacing prosthetic device that has all of the advantages of the prior art and none of the disadvantages.

It is another object of the present invention to provide a hip resurfacing prosthetic device that is held securely in place and reduces occurrences of periprosthetic loosening.

Another object of the present invention is to provide a hip resurfacing prosthetic device that does not utilize cement to fix its metallic cap to the outer surface of the femoral head.

Another object of the present invention is to provide a hip resurfacing prosthetic device that utilizes a femoral plate to provide additional stability to the prosthesis.

Yet another object of the present invention is to provide a hip resurfacing prosthetic device that promotes osseointegration between the femoral head and the metallic cap to promote additional securement therebetween.

Still yet another object of the present invention is to provide a hip resurfacing prosthetic device that does not require a complete ostectomy of the femoral head and neck.

Another object of the present invention is to provide a hip resurfacing prosthetic device that mimics the natural transfer of body load in a healthy hip joint and thereby prevents loosening of the prosthesis and periprosthetic fractures or periprosthetic osteoporosis.

Other objects, features and advantages of the present invention will become apparent from the following detailed description taken in conjunction with the accompanying drawings.

### BRIEF DESCRIPTIONS OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a cross-sectional view of the present invention affixed over a femoral head.

FIG. 2 shows a cross-sectional view of the metallic cap and the associated cannulated fixation means components of the present invention.

FIG. 3 shows a perspective view of the inner hemispherical surface of the metallic cap component of the present invention.

FIG. 4 shows a perspective view of the present invention demonstrating the angle between the cannulated fixation means and the femoral plate.

### DETAILED DESCRIPTION OF THE INVENTION

Reference is made herein to the attached drawings. Like reference numerals are used throughout the drawings to depict like or similar elements of the cementless hip resurfacing prosthesis. For the purposes of presenting a brief and clear description of the present invention, the preferred embodiment will be discussed as used for hip resurfacing procedures. The figures are intended for representative purposes only and should not be considered to be limiting in any respect.

The present invention generally comprises a metallic cap having a locking means and a femoral plate having a cannulated fixation means extending therefrom. The cannulated fixation means extends through the femoral head and neck, engaging with the locking means, and then a fixation screw is inserted through the cannulated fixation means into the locking means. The fixation screw secures the metallic cap to the femoral head and to the femoral plate, which provides additional stability for the device. The device is intended to be utilized in hip resurfacing procedures in order to provide a new articulating surface for the femoral head, thereby preventing additional degradation to the femoral head. The metallic cap articulates with a corresponding acetabular prosthesis, which is not depicted nor claimed in the present disclosure.

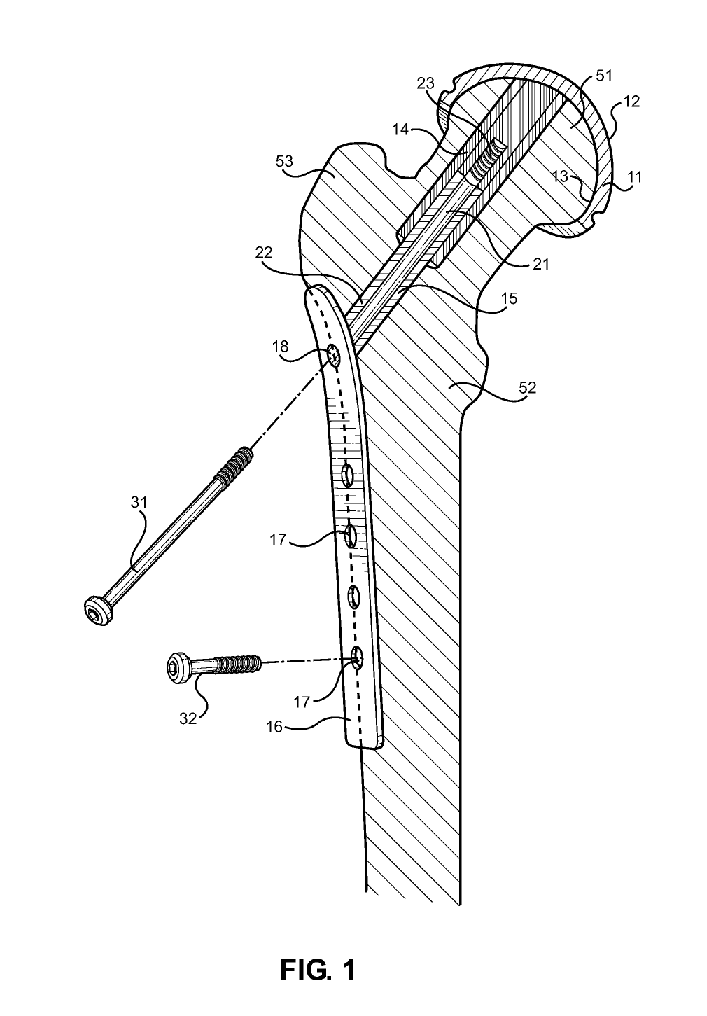

Referring now to FIG. 1, there is shown a cross-sectional view of the present invention affixed over a femoral head. The present invention comprises a metallic cap 11 with locking means 14 extending from its inner surface 13 and a femoral plate 16 having a cannulated fixation means 15 extending medially therefrom. The cannulated fixation means 15 has a first end 21, which is designed to engage with the locking means 14, and a second end 22, which is attached to the femoral plate 16. The present invention does not utilize cement in order to affix the metallic cap 11 to the femoral head because cement degrades over time, making it unsuitable for long term replacements, and furthermore can damage the femoral bone because the curing process for the cement is an exothermic reaction. The present invention instead relies upon the fixation screw 31 inserted through the cannulated fixation means 15 and the cortical screws inserted through the femoral plate 16 to firmly secure the metallic cap 11 in place and distribute the body load from the forces coming through the joint.

The metallic cap 11 consists of a thin-walled hemispherical shell having a convex outer hemispherical surface 12 and a concave inner hemispherical surface 13. The inner hemispherical surface 13 has a radius of curvature adapted to fit the anatomy of a femoral head with minimal surgical alteration made to the femoral head. Although the femoral head may need to be surgically shaped to the proper geometry to fit the metallic cap 11, the hip resurfacing procedure nonetheless avoids the extensive ostectomy procedure that is required for a total hip replacement. The outer hemispherical surface 12 is adapted to articulate with a corresponding acetabular surface, which is not claimed herein. The metallic cap 11 is preferably constructed from a cobalt or titanium-based alloy, or another suitable biocompatible material.

A locking means 14 is disposed along the inner hemispherical surface 13. The locking means 14 preferably comprises a base portion affixed to the inner hemispherical surface 13 and a cylindrical tubular portion that has a substantially smooth channel with a first diameter and a threaded channel 23 or aperture with a second, smaller diameter. The locking means 14 is adapted to accept the cannulated fixation means 15 and comprises a threaded channel 23 that is designed to engage with the complimentary threaded portion of the fixation screw 31. The inner diameter of locking means 14 is substantially equal to the outer diameter of the cannulated fixation means 15 and the locking means 14 and the cannulated fixation means 15 furthermore have a complimentary shape, allowing the cannulated fixation means 15 to slide into the locking means 14 and be held securely therein. The metallic cap 11 and femoral plate 16 components of the present invention are held securely together within the femoral head 51 and neck 52 and also held securely against the exterior of the femoral cortical bone by the fixation screw 31. The fixation screw 31 is inserted through the fixation aperture 18 on the femoral plate 16, into the channel of the cannulated fixation means 15, through the channel of the locking means 14, and engages with the complimentary threaded portion 23 of the locking means 14. Locking the metallic cap 11 to the lateral femoral surface helps to reduce to risk that the prosthetic metal cap 11 will slide or loosen during use.

The femoral plate 16 further has a plurality of cortical apertures 17 disposed along its length, through which a like number of cortical screws 32 may be inserted. The cortical screws 32 are applied directly through the cortical bone of the femur and lock the femoral plate 16 and the rest of the present invention to the femur. The cortical screws 32 also serve to distribute the forces placed on the femoral head 51 joint throughout the cortical bone. Femoral head prostheses that have components running through the inner cancellous bone run the risk of creating stress shielding and bone resorption issues as stresses are removed from the cortical bone and are transferred to the cancellous bone. Firmly affixing the present invention to the femoral cortical bone helps to ensure that the stresses are properly distributed and that the prosthesis does not act to weaken the bone over time.

The femoral plate 16 is a plate that is designed to affix to the anatomy of the lateral portion of the femur against the greater trochanter 53 or just below the greater trochanter 53, in alignment with the metallic cap 11 and the locking means 14 extending laterally therefrom. The femoral plate 16 is depicted as an elongated, substantially flat member with a square bottom end and an angled, rounded top end. The angled, rounded top portion is designed to be placed against the greater trochanter 53 to help support the present invention when in use. However, no claim is made as to the specific design or configuration of the femoral 16, except that it is designed to complement the anatomy of the exterior of the femoral shaft bone.

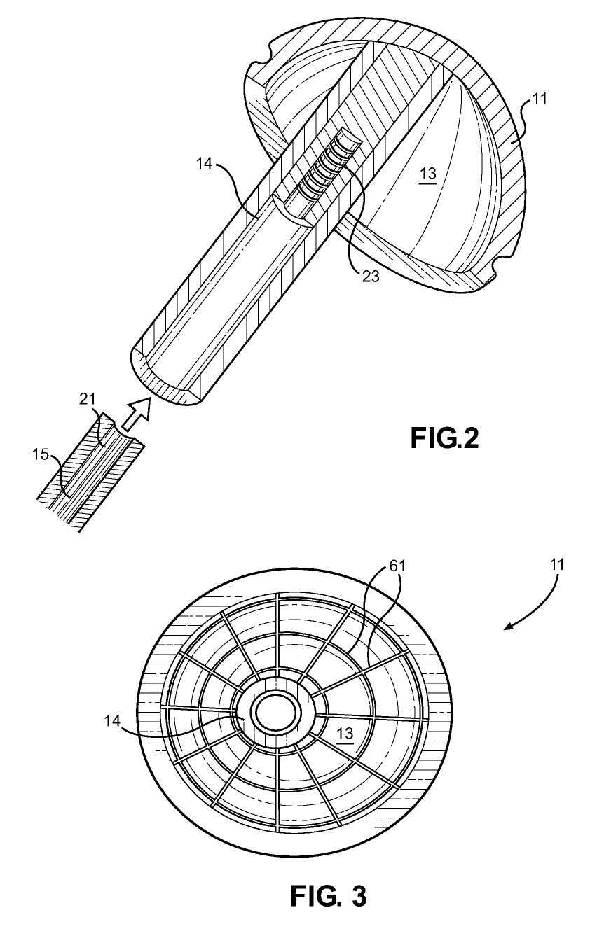

Referring now to FIG. 2, there is shown a cross-sectional view of the metallic cap and the associated cannulated fixation means components of the present invention. The locking means 14 extends laterally from the inner hemispherical surface 13 of the metallic cap, through the femoral head 51. The locking means 14 has a first portion, which has a first diameter and that is designed to accept the first end 21 of the cannulated fixation means 15, and a second threaded portion 23, which has a second diameter and is designed to engage with the complimentary threaded portion of the fixation screw.

When the present invention is installed onto the patient's femoral head, the metallic cap 11 substantially covers the patient's femoral head, creating a new articulating surface. A hole must be drilled through the femur from the femoral head to a measured location on the lateral portion of the greater trochanter or the femoral shaft. The locking means 14 extends through the pre-drilled hole in the femoral head and engages with the cannulated fixation means 15, which is inserted through the opposing end of the pre-drilled hole. When the locking means 14 and the cannulated fixation means 15 engage together, an uninterrupted channel is formed between the two components that allows the fixation screw to secure the femoral plate and the metallic cap 11 together.

Referring now to FIG. 3, there is shown a perspective view of the inner hemispherical surface of the metallic cap component of the present invention. The inner hemispherical surface 13 preferably has a plurality of osseointegration notches 61 extending laterally and longitudinally across its face to create a roughened or porous surface. No claim is made as to the precise orientation or number of the osseointegration notches. The osseointegration notches 61 are micro-rough surface grooves that provide space for the femoral bone to grow into the prosthesis, thereby encouraging interaction between the present invention and the femoral head. Increased bone-prosthesis interaction allows the present invention to be more firmly secured to the femoral head and therefore reduces the rate of loosening of the prosthesis and periprosthetic fracture. The outer hemispherical surface 12, on the other hand, is smoothed and polished in order to promote optimal articulation between the present invention and the corresponding acetabular surface.

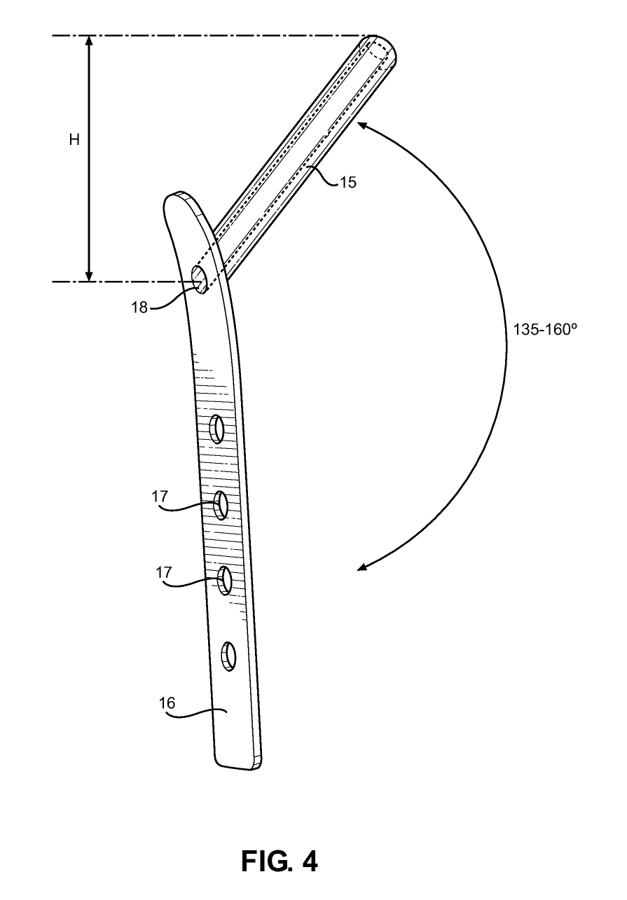

Referring now to FIG. 4, there is shown a perspective view of the present invention demonstrating the angle between the cannulated fixation means and the femoral plate. The angle between the cannulated fixation means 15 and the femoral plate 16 is preferably set at 135 to 160 degrees, depending upon the anatomy of the particular patient. This angle between the two components allows for the force to be transmitted from the femoral head, down the cannulated fixation means 15, and into the femoral plate 16 without any offset forces into the surrounding bone. If the angle between the two prosthetic components is outside of this range, then the stress from the joint on the prosthesis may create a shearing force on the femoral neck that could eventually result in periprosthetic fracture.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1. A cementless femoral head resurfacing prosthesis, comprising:
a metallic cap adapted to fit over a femoral head, said metallic cap having an inner surface and an outer surface;
a locking means extending from said inner surface, said locking means having a tubular portion and a threaded portion;
a femoral plate adapted to be fitted against a femoral shaft;
a tubular fixation means extending from said femoral plate at an angle;
a fixation screw;
said locking means and said fixation means designed to engage together and allow said fixation screw to pass therethrough and engage with said threaded portion, securely holding said femoral plate in place against a femoral shaft and holding said metallic cap in place against a femoral head.

2. The cementless femoral head resurfacing prosthesis of claim 1, further comprising:
a plurality of cortical apertures disposed on said femoral plate;
a plurality of cortical screws adapted to be secured to a femoral shaft through said cortical apertures, further securing said femoral plate to said femoral shaft.

3. The cementless femoral head resurfacing prosthesis of claim 1, wherein said metallic cap is composed of a cobalt-based alloy.

4. The cementless femoral head resurfacing prosthesis of claim 1, wherein said metallic cap is composed of a titanium-based alloy.

5. The cementless femoral head resurfacing prosthesis of claim 1, wherein said fixation means is disposed at an angle between 135 and 160 degrees relative to said femoral plate.

6. The cementless femoral head resurfacing prosthesis of claim 1, wherein said inner surface is roughened to encourage osseointegration with said metallic cap.

7. The cementless femoral head resurfacing prosthesis of claim 1, further comprising a plurality of osseointegration notches disposed on said inner surface of said metallic cap.

8. A cementless femoral head resurfacing prosthesis, comprising:
a metallic cap adapted to fit over a femoral head, said metallic cap having an inner surface and an outer surface;
said inner surface having a plurality of osseonintegration notches disposed on said inner surface of said metallic cap;
a locking means extending from said inner surface, said locking means having a tubular portion and a threaded portion;
a femoral plate adapted to be fitted against a femoral shaft;
a tubular fixation means extending from said femoral plate at an angle, said fixation means disposed at an angle between 135 and 160 degrees relative to said femoral plate;
a fixation screw;
said locking means and said fixation means designed to engage together and allow said fixation screw to pass therethrough and engage with said threaded portion, securely holding said femoral plate in place against a femoral shaft and holding said metallic cap in place against a femoral head;
a plurality of cortical apertures disposed on said femoral plate;
a plurality of cortical screws adapted to be secured to a femoral shaft through said cortical apertures, further securing said femoral plate to said femoral shaft.

9. The cementless femoral head resurfacing prosthesis of claim 8, wherein said metallic cap is composed of a cobalt-based alloy.

10. The cementless femoral head resurfacing prosthesis of claim 8, wherein said metallic cap is composed of a titanium-based alloy.
